(19) United States
(12) Patent Application Publication
(10) Pub. No.: US 2015/0072386 A1
(43) Pub. Date: Mar. 12, 2015

(54) METHOD FOR PRODUCING ORGANIC ACID

(71) Applicant: ASAHI GLASS COMPANY, LIMITED, Chiyoda-ku (JP)

(72) Inventors: Tatsuhiro . NOGAMI, Chiyoda-ku (JP); Nobuyuki KASAHARA, Chiyoda-ku (JP); Tsubasa TAKASUGI, Chiyoda-ku (JP)

(73) Assignee: ASAHI GLASS COMPANY, LIMITED, Chiyoda-ku (JP)

(21) Appl. No.: 14/539,742

(22) Filed: Nov. 12, 2014

Related U.S. Application Data
(63) Continuation of application No. PCT/JP2013/006921, filed on Jul. 12, 2013.

(30) Foreign Application Priority Data
Jul. 23, 2012 (JP) 2012-162439
Jul. 23, 2012 (JP) 2012-162440

(51) Int. Cl. C07C 51/48; C12P 7/56

(52) U.S. Cl. CPC C07C 51/48; C12P 7/56
USPC 435/139; 435/136; 435/146; 435/145; 435/142

(57) ABSTRACT

To provide a method for producing an organic acid, whereby the desired organic acid can be efficiently recovered without necessity for adjusting the pH to a neutral level in the fermentation step. The method for producing an organic acid, comprises

a first step of producing an organic acid by fermentation to obtain a crude liquid containing the organic acid and having a pH of from 1 to 5, and

a second step of extracting the organic acid from the crude liquid containing the organic acid obtained in the first step by means of an extraction medium containing a C₁₀₋₃₀ diester compound and an alkylamine compound to obtain an extract (1).

### TECHNICAL FIELD

The present invention relates to a method for producing an organic acid, particularly to a method for efficiently extracting an organic acid produced by fermentation from the fermentation medium.

### BACKGROUND ART

Organic acids such as lactic acid, succinic acid, etc. have been used in various applications to medicines, agricultural chemicals, cosmetic products, etc. As a method for producing an organic acid, a fermentation method has been employed from long ago. Usually, an organic acid-producing bacterium is susceptible to fermentation inhibition by an organic acid to be produced, and therefore, in many cases, fermentation is carried out while adjusting the pH to a neutral level by adding an alkali to the fermentation medium. In such cases, the organic acid is recovered as a salt of the pH-adjusting agent. For example, lactic acid is extracted as ammonium lactate by means of a mineral acid and an alkylated amine (Patent Document 1). However, such a pH-adjusting operation is cumbersome and requires a step of returning the organic acid salt to the organic acid, thus leading to an increase of production costs.

Under the circumstances, it has been proposed to carry out fermentation by using a bacterium having acid resistance imparted so that fermentation can be conducted under an acidic condition, e.g. by using a transformant of an acid resistant microorganism as host (Patent Document 2).

In this case, the organic acid can be recovered in the form of an acid. As such a recovery method, a method for extraction with a mixture of a non-water miscible amine and a non-water miscible organic acid (Patent Document 3), a method for extraction with an oxygen-containing saturated heterocyclic compound (Patent Document 4) or a method for extraction with a solvent azeotropic with water (Patent Document 5) is known. In the first method, it is required to use a non-water miscible organic acid in order to conduct the extraction and back extraction at the same temperature. Further, the mixing ratio of the organic acid to the amine must be adjusted in a limited range, and such a range is required to be changed depending upon the organic acid to be produced, such being not practical. In the second method, tetrahydrofuran or the like is used as the oxygen-containing saturated heterocyclic compound, but with such a hydrophilic solvent, hydrophilic substances other than the organic acid contained in the fermentation medium may also be extracted. In the third method, as the solvent azeotropic with water, a lower alcohol such as methanol or ethanol is used, but in the purification step, such an alcohol for extraction has to be removed in a large amount, and further, there is a problem of esterification.

### PRIOR ART DOCUMENTS

### Patent Documents

Patent Document 1: JP-A-2010-539911

Patent Document 2: WO2011/021629

Patent Document 3: JP-B-59-40375

Patent Document 4: JP-A-8-337552

Patent Document 5: WO2007/114017

### DISCLOSURE OF INVENTION

### Technical Problem

Under the circumstances, the present invention is to provide a method for producing an organic acid, whereby the desired organic acid can be efficiently recovered without necessity for the pH adjustment in the fermentation step or without the above-mentioned various problems.

### SOLUTION TO PROBLEM

That is, the present invention provides a method for producing an organic acid, as defined in the following [1] to [11].

[1] A method for producing an organic acid, which comprises a first step of producing an organic acid by fermentation to obtain a crude liquid containing the organic acid and having a pH of from 1 to 5, and a second step of extracting the organic acid from the crude liquid containing the organic acid obtained in the first step by means of an extraction medium containing a C₁₀₋₃₀ diester compound to obtain an extract (1) containing the organic acid.
[2] The method according to [1], wherein the diester compound is a dialkyl ester of an aliphatic acid dicarboxylic acid.
[3] The method according to [1] or [2], wherein the diester compound is a diester compound selected from the group consisting of bis(2-ethylhexyl) fumarate, bis(2-ethylhexyl) sebacate, bis(2-ethylhexyl) itaconate, bis(2-ethylhexyl) azelate and bis(2-ethylhexyl) maleate.
[4] The method according to any one of [1] to [3], wherein the extraction medium further contains an alkylamine compound.
[5] The method according to [4], wherein the alkylamine compound is a C₁₅₋₃₉ trialkylamine.
[6] The method according to [5], wherein the trialkylamine is a trialkylamine selected from the group consisting of trihexylamine, trioctylamine, tridecylamine and tridodecylamine.
[7] The method according to any one of [4] to [6], wherein the volume ratio of the alkylamine compound/the ester compound in the extraction medium is from 0.6/1 to 9/1.
[8] The method according to any one of [1] to [7], which further includes a third step of extracting the organic acid from the extract (1) by means of water to obtain an extract (2) containing the organic acid.
[9] The method according to [8], wherein the third step is carried out at a temperature of from 60 to 90° C.
[10] The method according to any one of [1] to [9], wherein the second step is carried out at a temperature of from 0 to 40° C.

### ADVANTAGEOUS EFFECTS OF INVENTION

According to the present invention, it is unnecessary to carry out the pH adjustment in the fermentation step. Further, by the combination of the ester compound and the alkylamine compound, it is possible to obtain the organic acid selectively with high efficiency. That is, it is possible to obtain the organic acid selectively by a simple operation without substantially extracting components (particularly glucose) in the fermentation medium.

### DESCRIPTION OF EMBODIMENTS

In the present invention, fermentation means to let a microorganism produce a desired compound (organic acid).

In the present invention, the organic acid may be an organic compound having a carboxy group, and for example, lactic acid, 3-hydroxypropionic acid, pyruvic acid, malonic acid, succinic acid, malic acid, fumaric acid, maleic acid, glutaric acid, adipic acid, etc. may be mentioned. Among them, lactic acid is preferred, since it has a wide range of applications. Such an organic acid may be a D-isomer, a L-isomer or a DL-isomer, or it may form an oligomer i.e. a polymer having a polymerization degree of from about 2 to 15.

So long as an organic acid is produced by a microorganism, the fermentation in the first step may be homo-type fermentation wherein only the organic acid is produced, or hetero-type fermentation wherein ethanol, etc. are produced in addition to the organic acid.

The microorganism may be a wild type or a gene-modified type. The wild type microorganism may, for example, be a lactic acid fermenter such as Streptococcus, Pediococcus, Leuconostoc or Lactobacillus; or a succinic acid fermenter such as Anaerobiospirillum or Corynebacterium.

The gene-modified type microorganism may be a gene-modified lactic acid-producing yeast, and for example, a transformant of Schizosaccharomyces pombe as host, wherein a lactic acid dehydrogenase gene is integrated, and a part of gene cluster encoding pyruvic acid decarboxylase of the host is deleted or deactivated (Patent Document 2), a transformant of an acid resistant microorganism such as a yeast in Genus Saccharomyces, as host, wherein a gene encoding a lactic acid dehydrogenase to the acid resistant microorganism is introduced (JP-A-2001-204464), and Saccharomyces cerevisiae (budding yeast), wherein a gene encoding a lactic acid dehydrogenase is introduced, and a gene encoding pyruvic acid decarboxylase 1 is deleted or deactivated (JP-A-2008-48726), may be mentioned. Among them, the transformant of Schizosaccharomyces pombe as host, wherein a lactic acid dehydrogenase gene is integrated, and a part of gene cluster encoding pyruvic acid decarboxylase of the host is deleted or deactivated, is preferred in that lactic acid is thereby produced with high productivity without necessity to adjust the pH to a neutral level.

The fermentation medium to be used for the fermentation is not particularly limited and may contain fundamental inorganic salts of e.g. Na, K, etc. and a carbon source, which are suitable for the production of a desired organic acid. Further, as the case requires, it may contain a nitrogen source and components such as an amino acid, etc. The fermentation medium may be a natural, synthetic or semi-synthetic fermentation medium. The carbon source may, for example, be a saccharide such as glucose, fructose, sucrose or maltose. The nitrogen source may, for example, be ammonia, an ammonium salt of an inorganic or organic acid such as ammonium chloride or ammonium acetate, peptone, casamino acids, or a yeast extract. The inorganic salt may, for example, be magnesium phosphate, magnesium sulfate, sodium chloride, or potassium dihydrogen phosphate. Further, a fermentation accelerator such as proteolipid may be incorporated.

It is preferred to use glucose as the saccharide. The glucose concentration in a fermentation medium (100 mass %) at the initial stage of fermentation is preferably at least 1 mass %, more preferably from 1 to 25 mass %, further preferably from 2 to 16 mass %. Since the glucose concentration decreases by fermentation, it is preferred to continue fermentation by adding glucose as the case requires. At the terminal stage of fermentation, the glucose concentration may be at most 1 mass %. Further, in a case where fermentation is carried out continuously by circulating the fermentation medium while separating the organic acid, it is preferred to maintain the above glucose concentration. When the glucose concentration is made to be at least 2 mass %, the productivity of the organic acid is more improved. When the glucose in the fermentation medium is made to be at most 16 mass %, the production efficiency of the organic acid is more improved.

Further, in order to increase the productivity, it is preferred to carry out high density fermentation. In the high density fermentation, the initial cell concentration of the transformant in the fermentation medium is made to be preferably from 0.1 to 50 g/L, more preferably from 0.2 to 40 g/L, as a value calculated by weight of dry cell. By increasing the initial cell concentration, high productivity can be accomplished in a short time. However, if the initial cell concentration is too high, a problem such as agglomeration of cells or a decrease in the purification efficiency is likely to result. Further, the cell concentration shown in Examples given hereinafter, is a value calculated from the absorbance of light at a wavelength of 660 nm (OD660) measured by a visible ultraviolet spectrometer V550 manufactured by JASCO corporation. OD=1 at 660 nm corresponds to 0.2 g/L of the dry weight of yeast and 0.8 g/L of the wet weight.

For the fermentation, a known fermentation method may be used, and for example, the fermentation may be conducted by circulation fermentation or stirring fermentation. The fermentation temperature is preferably from 23 to 37° C. The fermentation time may suitably be determined. The fermentation may be batch fermentation or continuous fermentation. For example, after carrying out fermentation by batch fermentation, the cells may be separated from the fermentation medium to obtain the fermentation medium containing the organic acid. Whereas, in the continuous fermentation method, for example, a part of the fermentation medium is withdrawn from the fermentation vessel during fermentation; from the withdrawn fermentation medium, the organic acid is separated; and to the remaining fermentation medium having the organic acid removed, glucose or a fresh fermentation medium is added, and the mixture is returned to the fermentation vessel. This operation is repeated to continuously carry out fermentation. By such continuous fermentation, the productivity of the organic acid is further improved.

The organic acid-containing crude liquid which contains the produced organic acid, has a pH of from 1 to 5, preferably from 1.5 to 4, particularly preferably from 1.5 to 3.5. In the method for producing an organic acid of the present invention, it is preferred to employ a fermentation method whereby an organic acid can be produced without carrying out pH adjustment even when the pH becomes low due to accumulation of the organic acid in the fermentation medium. That is, it is preferred to employ a fermentation method wherein an organic acid can be produced by continuous fermentation whereby fermentation is continued even after the pH of the fermentation medium becomes low. In such a fermentation method, in order to increase the productivity of the organic acid, it is preferred to continue fermentation even after the pH of the fermentation medium becomes to be at most 3.5. Especially, the above-mentioned transformant of Schizosaccharomyces pombe is excellent in acid resistance, whereby fermentation can be continued without adjusting the pH of the fermentation medium containing the produced organic acid.

In the second step, from the crude liquid containing the organic acid obtained in the first step, the desired organic acid is extracted by means of an extraction medium containing an ester compound to obtain an extract (1). The crude liquid containing the organic acid may be subjected directly to extraction, but preferably, prior to the extraction, yeast cells may be separated by cell separation treatment such as centrifugal separation or filtration. Conditions for the centrifugal separation may, for example, be from 10 to 15 minutes at from 1,000 to 5,000 G. As a condition for the filtration, a filtration membrane having a nominal opening of from 0.1 to 2 μm may be used. A typical composition of the crude liquid containing the organic acid comprises, for example, from 50 to 120 g/L of the organic acid, from 0.5 to 20 g/L of saccharides and from 1 to 20 g/L of ethanol.

The ester compound is preferably at least one member selected from the group consisting of C₄₋₄₀ aliphatic esters and aromatic esters. So long as it is one having a number of carbon atoms within the above range, it has proper polarity and boiling point, whereby extraction efficiency is good, and the removal in the subsequent step is easy. The boiling point of the ester compound under ordinary pressure is preferably at least 250° C. The upper limit for the boiling point is not particularly limited, but is usually at most 400° C. However, in a case where there is no boiling point under normal pressure, and only a decomposition point is present, the decomposition point is preferably at least 250° C.

Among the above aliphatic esters and aromatic esters, as the ester compound of the present invention, an aliphatic ester is preferred. That is, it is preferred that each of the carboxylic acid residue and the alcohol residue in the ester compound is a residue of an aliphatic compound.

Ester compounds are classified into a monoester compound and a polyester compound depending upon the number of ester groups in one molecule. As the ester compound of the present invention, a polyester having from 2 to 4 ester groups is preferred, and a diester compound is more preferred. Particularly preferred is a C₁₀₋₃₀ aliphatic diester compound.

The diester compound may, for example, be a diester compound having one dicarboxylic acid residue and two monool residues, or a diester compound having two monocarboxylic acid residues and one diol residue. The ester compound of the present invention may be either one of them, but an aliphatic diester compound having a dicarboxylic acid residue and monool residues is more preferred. Particularly preferred is a diester compound having a saturated or unsaturated aliphatic dicarboxylic acid residue and alkanol residues.

A preferred ester compound in the present invention is a C₁₀₋₃₀ aliphatic diester compound. Among them, an aliphatic diester compound having a saturated or unsaturated aliphatic dicarboxylic acid residue and alkanol residues (i.e. alkyl groups) is more preferred. Further, the number of carbon atoms in the saturated aliphatic dicarboxylic acid residue (the number including carbon atoms of carbonyl groups) is preferably from 5 to 15, more preferably from 6 to 12. The number of carbon atoms in the unsaturated aliphatic dicarboxylic acid residue (the number including carbon atoms of carbonyl groups) is preferably from 4 to 8, more preferably from 4 to 6. The number of carbon atoms in the alkanol residue (i.e. alkyl group) is preferably from 2 to 12, more preferably from 6 to 10.

Specifically, for example, diethyl adipate, diisononyl adipate, diundecyl adipate, bis(2-ethylhexyl) adipate, diethyl pimelate, didecyl pimelate, dioctyl sebacate, bis(2-ethylhexyl) sebacate, dibutyl sebacate, diethyl azelate, dioctyl azelate, bis(2-ethylhexyl) azelate, dihexyl azelate, bis(2-ethylhexyl) dodecanedioate, dibutyl fumarate, dinonyl fumarate, bis(2-ethylhexyl) fumarate, dihexyl maleate, bis(2-ethylhexyl) maleate, dipropyl itanonate and bis(2-ethylhexyl) itaconate may be mentioned.

Among them, an ester compound selected from the group consisting of bis(2-ethylhexyl) fumarate, bis(2-ethylhexyl) sebacate, bis(2-ethylhexyl) itanonate, bis(2-ethylhexyl) azelate and bis(2-ethylhexyl) maleate, is most preferred.

The above extraction medium preferably further contains an alkylamine compound. As the alkylamine compound, a dialkylamine compound or a trialkylamine compound is preferred. As the dialkylamine compound, a compound having two C₃₋₁₅ alkyl groups is preferred. As the trialkylamine compound, a compound having three C₃₋₁₅ alkyl groups is preferred. The plurality of alkyl groups in one molecule of the dialkylamine compound or the trialkylamine compound may be different.

Such an alkylamine compound has good compatibility with the above ester compound and increases the efficiency for extraction of the desired organic acid to the ester compound. Further, in a case where extraction with water is further carried out as described later, it does not hinder the extraction of the organic acid to water.

The boiling point of the alkylamine compound under ordinary pressure is preferably at least 250° C. The upper limit for the boiling point is not particularly limited, but is usually at most 400° C. However, if there is no boiling point under ordinary pressure, and only a decomposition point is present, the decomposition point is preferably at least 250° C.

More preferably, a C₁₅₋₃₉ trialkylamine is used. For example, dibutylundecylamine, tripentylamine, dipentylundecylamine, trihexylamine, trioctylamine, trinonylamine, triundecylamine, tridecylamine and tridodecylamine may be mentioned. Among them, an alkylamine compound selected from the group consisting of trihexylamine, trioctylamine, tridecylamine and tridodecylamine is most preferred.

In the case of using the ester compound and the alkylamine compound in combination, their mixing ratio is preferably from 0.6/1 to 9/1, more preferably from 1/1 to 3/1, by volume ratio of the alkylamine compound/the ester compound. Within such a range, the extraction efficiency may be made high.

The extraction is conducted by mixing and contacting the organic acid-containing crude liquid and the extraction medium preferably at a temperature of from 0 to 40° C., more preferably from 0 to 30° C. The volume ratio of the organic acid-containing crude liquid/the extraction medium is adjusted to be from 0.5/1 to 2/1, preferably from 0.8/1 to 1.2/1. The extraction time may usually be from 1 to 10 minutes, although it depends on the mixing and contacting efficiency.

The extraction may be carried out by a batch operation or a continuous operation, but a continuous operation is preferred, since the extraction efficiency is thereby high and the energy required for the operation can easily be controlled to be low. The batch operation may, for example, be a shaking operation or a stirring operation. As the continuous operation, using a tray tower or a packed tower, concurrent extraction or countercurrent extraction may be carried out. It is preferred to conduct countercurrent extraction by means of a packed tower, since it is thereby easy to increase the extraction efficiency and to reduce the size of the apparatus.

The partition coefficient in the extraction by means of the extraction medium depends on the organic acid to be extracted and the extraction medium, but is preferably at least 0.2, more preferably at least 0.3. Further, the extraction rate is preferably at least 20%, more preferably at least 25%.

In the case of using the ester compound and the alkylamine compound in combination as the extraction medium, the partition coefficient in the extraction by means of the extraction medium is preferably at least 1.0, more preferably at least 1.4. Further, the extraction rate is preferably at least 50%, more preferably at least 60%.

On the other hand, the extraction rate of a carbon source (e.g. glucose) contained in the fermentation culture medium and an organic substance (e.g. ethanol) produced by fermentation is preferably at most 20%, more preferably at most 15%, further preferably at most 10%, particularly preferably at most 5%.

For example, by subjecting the extract (1) to distillation under reduced pressure, the extraction medium is removed to obtain the organic acid. Preferably, in a third step, the organic acid is extracted from the extract (1) by means of water to obtain an extract (2) containing the organic acid. The water preferably contains no salts, whereby the purity of the finally obtainable organic acid can easily be made high. That is, the water may be one of ion-exchanged water, distilled water and pure water. The extraction by means of water is carried out preferably from 60 to 90° C., more preferably from 70 to 90° C. The volume ratio of the extract (1)/water for extraction is from 0.5/1 to 2/1, preferably from 0.8/1 to 1.2/1. The extraction time depends on the mixing and contacting efficiency, but is usually from 3 to 6 hours.

The partition coefficient in the extraction by means of water may depend on the organic acid to be extracted, but is preferably at least 0.15, more preferably at least 0.2. Further, the extraction rate is preferably at least 15%, more preferably at least 17%.

In the case of using the ester compound and the alkylamine compound in combination as the extraction medium, the partition coefficient in the extraction by means of water is preferably at least 1.0, more preferably at least 1.4. Further, the extraction rate is preferably at least 50%, more preferably at least 60%.

The third step is preferably conducted continuously from the second step, but may be conducted in a batch system. By removing water from the extract (2) obtainable in the third step, by e.g. distillation under reduced pressure, it is possible to obtain the organic acid. At that time, the extract (2) may be purified by subjecting it to a known method, e.g. treatment with activated carbon.

Now, the present invention will be described in detail with reference to Examples and Comparative Example. However, it should be understood that the present invention is by no means restricted by these Examples.

<Preparation of crude liquid containing lactic acid>

Lactic acid fermentation was carried out by using genetically modified lactic acid-producing yeast ASP2782 (transformant of Schizosaccharomyces pombe as host, wherein a lactic acid dehydrogenase gene is integrated, and gene pdc-2 encoding pyruvic acid decarboxylase of the host is deleted) prepared in Ex. 3 of Patent Document 2 (WO2011/021629). The transformant was inoculated in a D12 liquid culture medium (glucose 12%) and fermented for 20 hours under conditions of a temperature of 32° C. and a shaking rate of 100 rpm to obtain a fermented liquid (pH2.3) having a lactic acid concentration of 85.7 g/L. The fermented liquid was subjected to centrifugal separation (12000 G, 5 minutes) to obtain a supernatant as a lactic acid-containing crude liquid.

### Examples 1 to 6

### <Extract (1)>

The obtained lactic acid-containing crude liquid and each ester compound shown in Table 1 were mixed in a volume ratio of 1:1. The obtained mixed liquid was held at 25° C. for 15 minutes, then shaken for 1 minute and then centrifugally separated (3000 G, 5 minutes) into an organic phase and an aqueous phase. The aqueous phase was removed to obtain an extract (1). The lactic acid concentration in the extract (1) was measured by a high performance liquid chromatograph (HPLC) method (Agilent 1100, manufactured by Agilent Technologies; column: TSKgelOApak-A, manufactured by Tosoh Corporation), to obtain an extraction rate and an extraction partition coefficient. Further, in Example 1, the extraction rates of glucose and ethanol obtained by the HPLC method in the same manner were 2.3% and 14.8%, respectively.

### <Extract (2)>

To the extract (1), ion-exchanged water in the same volume amount as the extract (1) was added, followed by stirring at 80° C. for 5 hours, and then centrifugally separated into an organic phase and an aqueous phase. The organic phase was removed to obtain an extract (2). The lactic acid concentration in the extract (2) was measured by the HPLC method to obtain an extraction rate and an extraction partition coefficient. The results are shown in Table 1.

### Examples 7 to 12

### <Extract (1)>

The obtained lactic acid-containing crude liquid, each ester compound shown in Table 2 and tri-n-octylamine were mixed in a volume ratio of 5:2:3. The obtained mixed liquid was held at 25° C. for 15 minutes, then shaken for 1 minute and then centrifugally separated (3000 G, 5 minutes) into an organic phase and an aqueous phase. The aqueous phase was removed to obtain an extract (1). The lactic acid concentration in the extract (1) was measured by the HPLC method to obtain an extraction rate and an extraction partition coefficient. Further, in Example 7, the extraction rates of glucose and ethanol obtained by the HPLC method in the same manner, were 3% and 5%, respectively.

### <Extract (2)>

To the extract (1), ion-exchanged water in the same volume amount as the extract (1) was added, followed by stirring at 80° C. for 5 hours, and then centrifugally separated into an organic phase and an aqueous phase. The organic phase was removed to obtain an extract (2). The lactic acid concentration in the extract (2) was measured by the HPLC method to obtain an extraction rate and an extraction partition coefficient. The results are shown in Table 2.

### Example 13

A test was conducted in the same manner as in Example 10 except that in Example 10, instead of tri-n-octylamine, tri-n-decylamine was used. The results are shown in Table 2.

### Comparative Example 1

Lactic acid was extracted and the extraction rates of lactic acid, glucose and ethanol were obtained in the same manner as for the above extract (1), except that the lactic acid-containing crude liquid and tetrahydrofuran were mixed in a ratio of 1:1, whereby the lactic acid extraction rate was 87.6%, the glucose extraction rate was 26.2%, and the ethanol extraction rate was 87.5%.

TABLE 1

| | | Extract (1) | | Extract (2) | |
|---|---|---|---|---|---|
| Ex- | | Extraction | Extraction | Extraction | Extraction |
| ample | | rate | partition | rate | partition |
| No. | Ester compound | (%) | coefficient | (%) | coefficient |
| 1 | Bis(2-ethylhexyl) | 25.8 | 0.35 | 17.8 | 0.22 |
| | fumarate | | | | |
| 2 | Bis(2-ethylhexyl) | 24.2 | 0.32 | 17.9 | 0.22 |
| | sebacate | | | | |
| 3 | Bis(2-ethylhexyl) | 25.8 | 0.35 | 16.1 | 0.19 |
| | itaconate | | | | |
| 4 | Bis(2-ethylhexyl) | 26.6 | 0.36 | 16.2 | 0.19 |
| | azelate | | | | |
| 5 | Bis(2-ethylhexyl) | 21.5 | 0.35 | 20.5 | 0.26 |
| | maleate | | | | |
| 6 | Dihexyl azelate | 20.3 | 0.25 | 21.0 | 0.27 |

TABLE 2

| | | Extract (1) | | Extract (2) | |
|---|---|---|---|---|---|
| Ex- | | Extraction | Extraction | Extraction | Extraction |
| ample | Ester compound + | rate | partition | rate | partition |
| No. | amine compound | (%) | coefficient | (%) | coefficient |
| 7 | Bis(2-ethylhexyl) | 68.24 | 2.15 | 68.82 | 2.21 |
| | fumarate + tri-n- | | | | |
| | octylamine | | | | |
| 8 | Bis(2-ethylhexyl) | 70.46 | 2.39 | 70.01 | 2.33 |
| | sebacate + tri-n- | | | | |
| | octylamine | | | | |
| 9 | Bis(2-ethylhexyl) | 70.97 | 2.44 | 66.18 | 1.96 |
| | itaconate + tri-n- | | | | |
| | octylamine | | | | |
| 10 | Bis(2-ethylhexyl) | 72.36 | 2.62 | 68.45 | 2.17 |
| | azelate + tri-n- | | | | |
| | octylamine | | | | |
| 11 | Bis(2-ethylhexyl) | 72.79 | 2.67 | 59.16 | 1.45 |
| | maleate + tri-n- | | | | |
| | octylamine | | | | |
| 12 | Dihexyl azelate + | 66.82 | 2.01 | 60.48 | 1.53 |
| | tri-n-octylamine | | | | |
| 13 | Bis(2-ethylhexyl) | 64.39 | 1.81 | 72.74 | 2.67 |
| | azelate + tri-n- | | | | |
| | decylamine | | | | |

As shown in Tables 1 and 2, in Examples 1 to 13, it was possible to extract lactic acid at a high extraction rate by each of the extraction by means of the ester compound, etc. and the extraction by means of water. Whereas, in Comparative Example 1, the contents of glucose and ethanol became high.

### INDUSTRIAL APPLICABILITY

According to the method for producing an organic acid of the present invention, it is possible to obtain, with high efficiency, an extract containing substantially no components in the fermentation medium, without necessity for adjusting the pH to a neutral level in the fermentation step.

This application is a continuation of PCT Application No. PCT/JP2013/069210, filed on Jul. 12, 2013, which is based upon and claims the benefit of priority from Japanese Patent Application No. 2012-162439 filed on Jul. 23, 2012 and Japanese Patent Application No. 2012-162440 filed on Jul. 23, 2012. The contents of those applications are incorporated herein by reference in their entireties.

What is claimed is:

1. A method for producing an organic acid, which comprises
a first step of producing an organic acid by fermentation to obtain a crude liquid containing the organic acid and having a pH of from 1 to 5, and
a second step of extracting the organic acid from the crude liquid containing the organic acid obtained in the first step by means of an extraction medium containing a C₁₀₋₃₀ diester compound to obtain an extract (1) containing the organic acid.

2. The method according to claim 1, wherein the diester compound is a dialkyl ester of an aliphatic acid dicarboxylic acid.

3. The method according to claim 1, wherein the diester compound is a diester compound selected from the group consisting of bis(2-ethylhexyl) fumarate, bis(2-ethylhexyl) sebacate, bis(2-ethylhexyl) itaconate, bis(2-ethylhexyl) azelate and bis(2-ethylhexyl) maleate.

4. The method according to claim 1, wherein the extraction medium further contains an alkylamine compound.

5. The method according to claim 4, wherein the alkylamine compound is a C₁₅₋₃₉ trialkylamine.

6. The method according to claim 5, wherein the trialkylamine is a trialkylamine selected from the group consisting of trihexylamine, trioctylamine, tridecylamine and tridodecylamine.

7. The method according to claim 4, wherein the volume ratio of the alkylamine compound/the ester compound in the extraction medium is from 0.6/1 to 9/1.

8. The method according to claim 1, which further includes a third step of extracting the organic acid from the extract (1) by means of water to obtain an extract (2) containing the organic acid.

9. The method according to claim 8, wherein the third step is carried out at a temperature of from 60 to 90° C.

10. The method according to claim 1, wherein the second step is carried out at a temperature of from 0 to 40° C.
